(19) United States
(12) Patent Application Publication
(10) Pub. No.: US 2008/0014658 A1
(43) Pub. Date: Jan. 17, 2008

(54) WEB MATERIAL WITH A COATING ALLOWING VERY RAPID SPREADING AND/OR TRANSPORT OF FLUIDS

(75) Inventors: Ingo Neubert, Norderstedt (DE); Maren Klose, Seevetal (DE)

Correspondence Address: NORRIS, MCLAUGHLIN & MARCUS, P.A., 875 THIRD AVE, 18TH FLOOR, NEW YORK, NY, 10022, US

(73) Assignee: tesa Aktiengesellschaft, Hamburg (DE)

(21) Appl. No.: 11/550,984

(22) Filed: Oct. 19, 2006

(30) Foreign Application Priority Data
Jul. 13, 2006 (DE) 10 2006 032 667.9

(51) Int. Cl. G01N 33/558

(52) U.S. Cl. 436/514

(57) ABSTRACT

Material in web form intended in particular for use in a diagnostic strip and composed of a carrier material having on one or both sides a coating comprising at least one surfactant
which is non-ionic,
which possesses a critical micelle concentration (CMC) of 0.1 g/l or less, and
whose 0.1% strength by weight aqueous solution has a surface tension of <30 mN/m.

The present invention relates to a material in web form that allows rapid spreading and/or very rapid transport of biological fluids such as blood, urine, saliva or cell fluid, for example.

### BACKGROUND OF THE INVENTION

In modern medical diagnosis, strips referred to as diagnostic test strips, or biosensors, are being used for an increasingly large number of analytical test strips. These strips or biosensors can be used, for example, to determine the level of glucose, cholesterol, proteins, ketones, phenylalanine or enzymes in biological fluids such as blood, saliva and urine.

The most frequently encountered diagnostic test strips are those used for determining and monitoring the blood sugar level in diabetics. Roughly 175 million people worldwide suffer from diabetes mellitus type 1 and type 2. The trend of this condition is rising. Patients affected by this incurable disease have to monitor their blood sugar level up to five times a day in order to obtain the best match between the dosage of the medication (insulin) and the consumption of food. In the event of an excessive blood sugar level, as a consequence of non-monitoring, considerable health-related damage is likely, and may even lead to death (diabetic shock as a result of undersupply or even oversupply of sugar). Until a few years ago, diabetics relied on the support of medical staff in order to determine the blood sugar level. To make monitoring blood sugar level as simple as possible a test was developed which enables the diabetic to determine his or her own blood sugar level with a minimum of effort and without reliance on medical staff.

To determine the blood sugar level the tester has to apply a drop of blood to a diagnostic test strip. During this procedure the diagnostic test strip is located in a read device or evaluation device. Following a reaction time or response time, the evaluation device indicates the current blood sugar level. Read or evaluation devices of this kind are described for example in U.S. Pat. No. 5,304,468 A, in EP 1 225 448 A1 and in WO 03/08091 A1.

One of the first patents in the field of test strips appeared back in 1964. U.S. Pat. No. 1,073,596 A describes a diagnostic test and the test strips for analysing biological body fluids especially for the purpose of determining blood sugar. The diagnostic test functions via the determination of a colour change which is triggered by an enzyme reaction.

Determining a change in the concentration of a dye (photometric method) is still a method used today in the determination of blood sugar using diagnostic test strips. The enzyme glucose oxidase/peroxidase reacts with the blood sugar. The hydrogen peroxide formed then reacts with an indicator, o-tolidene for example, which leads to a colour reaction. This alteration in colour can be monitored by calorimetric methods. The degree of colour change is directly proportional to the concentration of blood sugar. The enzyme in this case is located on a fabric.

This method is described for example in EP 0 451 981 A1 and in WO 93/03673 A1.

The modern development of diagnostic test strips is aimed at reducing the measurement time between the application of the blood to the test strip and the appearance of the result. The measurement time, or the time between application of the blood to the biosensor and display of the result, is dependent not only the actual time of the enzyme reaction and the ensuing reactions but also, to a considerable extent, on how rapidly the blood is transported within the biosensor from the point at which it is applied to the reaction site, in other words to the enzyme.

One of the ways in which the measuring time is reduced is by the use of hydrophilicized nonwovens or fabrics as in U.S. Pat. No. 6,555,061 B, in order to transport the blood more rapidly to the reaction site (enzyme). The measuring method is identical with that described in EP 0 451 981 A1. Surface-modified fabrics with a wick effect for the biological fluid are described in WO 93/03673 A1, WO 03/067252 A1 and US 2002/0102739 A1. In US 2002/0102739 A1 a blood transport of 1.0 mm/s is achieved through plasma treatment of the fabric. When fabrics are used to transport the biological test fluid, such as blood, for example, it is nevertheless the case that a chromatography effect is observed; in other words, the individual constituents, such as cells, are separated from the liquid constituents. The chromatography effect is explicitly exploited in WO 03/008933 A2 for the separate analysis of the blood constituents.

An onward development from the photometric measurement technique is the electrical determination of the change in oxidation potential and/or conductivity of an electrode coated with the enzyme. This method and a corresponding diagnostic test strip are described in WO 01/67099 A1. The diagnostic strip is constructed by the printing of various functional layers, such as electrical conductors, enzyme and hot-melt adhesive, onto the base material, which is made of polyester, for example. Subsequently, by means of thermal activation of the adhesive, a hydrophilic film, not described in any greater detail, is laminated on. The purpose of this hydrophilic film, here again, is to transport the blood to the measuring cell.

With this construction there is no need for fabric or nonwoven to transport the blood. The advantage of this construction and also of the new measurement technique is that the blood sugar level can be measured with a very much smaller volume of blood, of around 0.5 to 3 μl, and in a shorter measuring time of 3 to 5 seconds.

In principle the wettability can be improved and hence the rate of transport of the biological fluid within the diagnostic strip increased, with an accompanying reduction in the measuring time, by means of coatings with polar polymers such as, for example, polyvinylpyrrolidone, polycaprolactam, polyethylene glycol or polyvinyl alcohol. The wettability or hydrophilicity of these coatings, however, is too low for the rapid transport of biological fluids and hence unsuitable for the desired application.

Another possibility lies in the chemical or physical modification of the surfaces. Standard techniques for achieving this include corona treatment and flame treatment. Such treatments, however, are not stable over time. The marked increase in surface energy that is achieved as a result of the surface treatment falls back to the original level after just a few days.

Etching the surface with a strong acid likewise increases the hydrophilicity. Industrial films have their surfaces etched using, for example, oxidizing acids such as chromosulphuric acid or potassium permanganate in conjunction with sulphuric acid. Polyester films (PET) are typically hydrolysed in the industry by chemical treatment with, for example, trichloroacetic acid or potassium hydroxide on their surface, as disclosed in WO 2005/111606 A1. With these techniques the wettability and surface tension are stable even after storage. However, the wetting properties are not homogeneous over the treated area. Further information on the surface treatment of films is found in “Polymer Surface”, F. Garbassi et al., John Wiley Verlag 1998 (ISBN 0471971006).

Within the literature there are descriptions of the use of surfactants, known to the skilled person as interface-active substances, for the purpose of improving the wettability. Surfactants are molecules or polymers which are composed of an apolar/hydrophobic moiety (tail) and of a polar/hydrophilic group (head). To improve the wettability of surfaces the surfactants are added to the aqueous fluid. The surfactant brings about a reduction in the surface tension of the aqueous fluid at the interfaces (liquid/solid and liquid/gaseous). This effect of improving the wettability of the surfaces is measurable as a reduction in the contact angle. The skilled person makes a distinction between anionic, cationic, amphoteric and nonionic surfactants. The hydrophobic tail of surfactants may be composed of linear or branched alkyl, alkylbenzyl, perfluorinated alkyl or siloxane groups. Possible hydrophilic head groups are anionic salts of carboxylic acids, phosphoric acids, phosphonic acids, sulphates and sulphonic acids, cationic ammonium salts or nonionic polyglycosides, polyamines, polyglycol esters, polyglycol ethers, polyglycol amines, polyfunctional alcohols or alcohol ethoxylates (Ullmann's Encyclopaedia of Industrial Chemistry, Vol. A25, 1994, p. 747).

DE 102 34 564 A1 describes a biosensor which is composed of a planar sensor or test strip and of a compartmented reaction and measurement chamber attachment produced by embossing a PVC film. The sample reception channel and the measurement chamber are furnished with a hydrophilic fabric or a surfactant for the transport of the biological fluid. A very similar electrochemical sensor is described in U.S. Pat. No. 5,759,364 A1. The sensor is composed of a printed base plate and of an embossed outer film of PET or polycarbonate. In this case the measuring chamber is coated with a polyurethane ionomer for accelerated fluid transport.

A number of publications mention the use of hydrophilic materials such as fabrics (DE 30 21 166 A1), membranes (DE 198 40 008 A1) and films (EP 1 358 896 A1, WO 01/67099 A1), albeit without giving further details of the hydrophilic coatings.

DE 198 15 684 A1 describes an analytical aid composed of a zone of active capillarity, an adhesive tape diecut, and a cover film of active capillarity. The cover film of active capillarity possesses hydrophilic surface properties which are achieved by vapour-coating it with aluminium and by subsequent oxidation.

US 2005/0084681 A1 discloses a surface having a hydrophilic coating. This coating is composed of a surfactant, preferably a nonionic alcohol ethoxylate, and a stabilizer, preferably an alkylbenzyl sulphonate. The ratio of surfactant to stabilizer in the dried coating is one to three parts of surfactant to 0.25 to 2.5 parts of stabilizer. The stabilizer, which is preferably an anionic surfactant, ensures long-term stability for the hydrophilic coating. The stabilizing effect is brought about by the stabilizer having a melting temperature >25° C., preferably >45° C., as a result of which the vapour pressure is reduced (evaporation of the surfactant combination is reduced) and hence the long-term stability of the hydrophilic coating is attained.

Nowadays there are already hydrophilic films available commercially for use in medical diagnostic strips, examples being the products 9962 and 9971 from 3M Inc., whose use is shown in US 2002/0110486 A1 and EP 1 394 535 A1. These products feature are a polyester film which is provided on either one side or both sides with a hydrophilic coating. This coating consists of a polyvinylidene chloride coating comprising a surfactant based on an alkylbenzylsulphonate. The surfactant is required first to migrate to the surface of the coating before the hydrophilic surface properties can be developed. A detailed analysis shows that these products, while suitable for the transport of biological fluids in diagnostic test strips, exhibit significant deficiencies in respect of homogeneity and transport rate.

Further commercially available hydrophilic films include the products ARflow® 90128 and ARflow® 90469 from Adhesives Research Inc., whose use is shown in U.S. Pat. No. 5,997,817 A1. These products consist of polyester film which is coated with a thermoplastic copolyester, with the addition of a sulphosuccinate-based surfactant. The mode of action is analogous to that of the above-described 3M Inc. products. In order to avoid inhomogeneities, considerably larger amounts are added here, about 2% to 3% of the surfactant. The consequence of this, however, is a waxlike surface to the hydrophilic coating. It is not possible to achieve sufficient bond strength with pressure-sensitive adhesive tapes on this coating (see layer 2 in FIG. 1). In the production operation for the test strips, this leads frequently to the delamination of the assembly.

The diagnostic test strips described are produced in the majority of cases by means of a discontinuous sequence of coating steps and laminating steps. The base materials used are sheets of a 200 to 500 μm thick film of polyvinyl chloride, polyester or polycarbonate measuring about 400×400 mm.

For sometime now there have also been attempts to produce the diagnostic strips in continuous processes. The coating and laminating steps are commonly followed by a series of slitting operations. In view of the small size of the diagnostic strips, of around 20 mm×5 mm, a very high degree of precision is necessary in the coating, laminating and slitting operations. Slitting to form the diagnostic strips is typically accomplished at very high cycle rates using slitting machines which come from, for example, Siebler GmbH or Kinematik Inc. In the course of the slitting operations it is possible for problems to occur to a considerable extent. If unsuitable materials are used which exhibit inadequate adhesion to one another in the course of lamination, a delamination of the layers in the slitting operation is a frequently observed phenomenon. The source of this inadequate adhesion may lie in an unsuitable adhesive, in other words one with inadequate bond strength, or in an unsuitable bonding substrate, and/or in an unsuitable coating of the bonding substrate, as in the case of ARflow® 90128 and ARflow® 90469 from Adhesives Research Inc.

It is an object of the present invention to provide a material in web form which has a coating and which in accordance with the requirements for use in diagnostic test strips is suitable for their construction, and which in particular ensures rapid transport of the biological fluid within the measurement channel and also a very high assembly strength with pressure-sensitive adhesive tapes featuring high-shear-strength adhesives applied at a low rate. In this context it is necessary to ensure that the properties, and especially the wetting properties and transport properties, of the web material are retained even after a long period of storage.

### SUMMARY OF THE INVENTION

This object is achieved by means of a web material as specified in the main claim. The dependent claims provide advantageous developments of the subject matter of the invention. The invention further encompasses the possibility for use of the film of the invention in applications including in medical diagnostic strips for analysing biological fluids.

The invention accordingly provides a material in web form which is composed of a carrier material having on one or both sides a coating comprising at least one surfactant
which is non-ionic,
which possesses a critical micelle concentration (CMC) of 0.1 g/l or less, and
whose 0.1% strength by weight aqueous solution has a surface tension of <30 mN/m.

The surface coating of the material allows biological fluids, for example, to be transported rapidly, so that the material is suitable for medical applications such as diagnostic strips which are used to analyse biological fluids.

The characteristic property of the web material of the invention is its very good wettability for biological fluids such as blood, urine, saliva and cell fluid, leading to very wide and rapid spreading and hence to the transport of the fluids.

This property is reflected in:
a surface tension of at least 65 mN/m
a contact angle with water of less than 20°
an areal spread with 0.5 ml of water of at least 12 cm²
a spread rate of at least 100 mm²/s

The coating is additionally notable for very good compatibility with the detection reaction and enzyme reaction, which is to say that these reactions are unaffected by the coating or its ingredients. The high compatibility is manifested in the fact that the use of the material of the invention in a blood sugar measuring strip, for example, is not accompanied by observation of any change in the result of the measurement of the blood sugar level of the sample.

Furthermore, the coating is distinguished by very good ageing stability of the wetting and spreading properties. This ageing stability is manifested in the value for the areal spread of 0.5 ml of water on the coating, which even after storage at 40° C. for 10 weeks or at 70° C. for 4 weeks is at least 85% and preferably at least 90% of the original value prior to storage.

By spreading is meant the propagation of a fluid as a transport process on a solid phase (surface).

Surprisingly for the skilled person, these very good and ageing-resistant wetting and spreading properties can be obtained by virtue of the surfactant-containing coating of the invention.

### BRIEF DESCRIPTION OF THE DRAWINGS

FIG. 1 is a diagrammatic representation of a medical diagnostic test strip of the invention,

FIG. 2 is a graphical representation of the wetting of a solid as described by Young's equation.

### DETAILED DESCRIPTION

In the literature there are numerous investigations into the phenomenon of wettability and of the improvement in wettability through the use of surfactants.

The phenomenon of the wetting of a solid by a liquid is described by Young's equation (Eq. 1) (see FIG. 2 in this context)

γᵢ·cos 0=γₛ−γₛₗ Eq. 1

θ—contact angle (wetting angle)

γₗ—surface tension of the liquid

γₛ—surface tension of the solid

γₛₗ—interfacial tension between the liquid and the solid

If the contact angle θ is >>90°, liquid and solid are highly incompatible, and the surface of the solid is not wetted by the liquid. In the range from 90° to 20° there is a wetting of the solid's surface. At contact angles θ<20°, the surface tensions between liquid and solid are very similar, and there is very effective wetting of the solid's surface by the liquid. At contact angles θ<<20°(θ˜0°) the liquid spreads out over the solid's surface (see “Die Tenside”, Kosswig/Stache, Carl Hanser Verlag, 1993, ISBN 3-446-16201-1).

In each of these investigations it is the surfactant that is added to the liquid in order to obtain an improvement in the wetting properties of aqueous solutions for arbitrary substrates. There have to date been virtually no investigations in which the surfactant is applied directly to a surface in order to increase its wettability for arbitrary aqueous fluids. In the few such publications there are (see prior art) the surfactant-containing coating serves for hydrophilic modification of the interface between solid and fluid. In other words, the effect of wettability is achieved exclusively through an increase in the polarity of the surface (hydrophilic coating). For these or similar applications, therefore, polar ionic surfactants based on sulphosuccinate salts, such as sodium diethylhexylsulphosuccinate, for example, or on sulphonate salts, such as sodium octylbenzylsulphonate, for example, are used.

Surprisingly, and foreseeably for the skilled person, however, coatings with surfactants having a very low critical micelle concentration (CMC), a low surface tension of their aqueous solution, and very good water-solubility exhibit the best results with regard to wettability. Surprisingly, the factors necessary for the very effective spreading of liquids include not only the polar or hydrophilic modification of the surface but also a rapid distribution (dissolution) of the coating in the aqueous wetting fluid. The critical micelle concentration and the surface tension, and also the solubility, are consequently accorded considerable importance.

The critical micelle concentration indicates the minimum concentration of surfactant at which micelles form in the aqueous solution. At this concentration there is a change in a very wide variety of physical properties of the solution (Mukerjee et al., “Critical Micelle Concentration of Aqueous Surfactant Systems”, NSRDS-NBS 36 1971). For instance, the surface tension of the aqueous solution falls continually until the critical micelle concentration (CMC) is reached. At the point of critical micelle concentration the surface of the liquid is saturated with surfactant, and micelles begin to form in the solution. The surface tension then remains virtually constant as the surfactant concentration is increased (Addison et al., J. Chem. Soc. (1950), 3103).

Suitable coatings for the web material of the invention with very good wetting properties and spreading properties are coatings of at least one surfactant having a critical micelle concentration of less than 0.1 g/l, preferably less than 0.08 g/l and more preferably less than 0.06 g/l, the surfactant when added at 0.1% by weight lowering the surface tension of water (72.7 mN/m) to levels less than 30 mN/m and preferably less than 25 mN/m. The coating of at least one surfactant, and preferably only one surfactant having the properties specified above, and, with further preference, with at least one stabilizer in a concentration of not more than 1% by weight, based on the sum total of the surfactants in the coating, is distinguished by a contact angle with water of less than 20°, preferably less than 10°, by a surface tension of at least 65 mN/m, by a water spread rate of greater than 100 mm²/s, preferably greater than 150 mm²/s and more preferably greater than 200 mm²/s, the areal spread of 0.5 ml of water being at least 12 cm², preferably 15 cm² and more preferably 17 cm², and by a good strength of anchorage for adhesive tapes (no waxlike surface).

These properties exhibit little if any change even after storage periods of two years at 25° C. To assess the storage stability of the coatings an accelerated ageing is carried out at elevated temperatures. To assess the wetting properties, primarily the spread test (areal spread) is employed. The figure for the areal spread with 0.5 ml of water, after storage at 40° C. for 10 weeks or at 70° C. for 4 weeks, is at least 85% and preferably at least 90% of the original value prior to storage.

Without restriction, surfactants particularly suitable for the present invention are nonionic surfactants such as fatty alcohol ethoxylates, fluorosurfactants and silicone surfactants. Fatty alcohol ethoxylates are composed of linear or branched fatty alcohols which form the hydrophobic moiety of the surfactant. The hydrophilic moiety is composed of an ethoxylate which consists generally of three to twelve ethylene oxide units and is terminated with a methyl to butyl radical or hydrogen. The best wetting properties are achieved when the surfactant is composed of a branched C12-C18 fatty alcohol and an ethoxylate having three to five ethylene oxide units. There are only a few fatty alcohol ethoxylates suitable for the application of the invention. The reason for this is the poor water-solubility or inadequate interfacial activity (surface tension >30 mN/m for the 0.1% strength by weight aqueous solution) of the majority of the fatty alcohol ethoxylates. Examples of suitable fatty alcohol ethoxylate surfactants include Tego Surten® W111 from Degussa AG, Dynol® 604 and Surfynol® 465 from Air Products Inc., Triton® X-100 and Tergitol® 15-S from Dow Chemicals Inc.

Likewise suitable for the present invention are nonionic fluorosurfactants. These are composed of a hydrophobic perfluorinated alkyl radical, which in turn carries an ethoxylate, 1,2-propyleneoxy groups or other hydrophilic groups. Nonionic fluorosurfactants are notable for a very low surface tension of <25 mN/m for the 0.1% strength by weight aqueous solutions, and for very low critical micelle concentrations of <0.1 g/l. Exemplary structures of nonionic fluorosurfactants are as follows:

CF₃—(CF₂)ₘ—R

or CF₃—(CF₂)ₘ—CO—R

where R═—(O—CH₂—CH₂)ₙ—O—CH₃
or —(O—CH₂—CH₂)ₙ—O—H
or —(O—CH₂—CH₂(CH₃))ₙ—O—CH₃

with m=3 to 10 and n=2 to 8

For suitable non-ionic fluorosurfactants mention may be made for example of Fluorad® FC-4430 and FC-4432 from 3M Inc., Zonyl® FSO-100 from DuPont Inc. and Licowet® F 40 from Clariant AG. The use of fluorosurfactants in medical products, however, is objectionable on toxicological grounds and questionable from an economic standpoint; nevertheless, they meet the specifications set in accordance with the invention.

Particular preference for the present invention is given to using a coating comprising likewise preferably only one nonionic silicone surfactant. These surfactants are composed of a hydrophobic siloxane backbone. This backbone is composed typically of three to 100 repeating dimethylsiloxane units, which may be branched or linear and which are terminated by trimethylsiloxane groups. Suitable hydrophilic head groups are, again ethoxylates especially, composed of three to eight ethylene oxide units or 1,2-propyleneoxy units. Silicone surfactants typically have a very low surface tension of 25 to 20 mN/m for the 0.1% strength by weight aqueous solutions, and also a very low critical micelle concentration of <0.1 g/l. Particularly suitable for the present invention are silicone surfactants with short-chain siloxanes having, for example, the following structures:

(CH₃)₃Si—O—Si(CH₃)R—O—Si(CH₃)₃

or (CH₃)₃Si—O—Si(CH₃)R—O—Si(CH₃)₂—O—Si(CH₃)₃

or (CH₃)₃Si—O—Si(CH₃)R—(O—Si(CH₃)₂)ₙ—O—Si(CH₃)₃

with n=2 to 10

where R═X—(CH₂)₃—(O—CH₂—CH₂)ₙ—O—CH₃
or X—(CH₂)₃—(O—CH₂—CH₂)ₙ—O—H
or X—(CH₂)₃—(O—CH₂—CH₂(CH₃))ₙ—O—CH₃

with n=2 to 10; X spacer, linking unit

Examples of silicone surfactants suitable for application according to the invention include Q2-5211 and Sylgard® 309 from Dow Corning Inc., Silwert® L-77 from GE Silicones Inc., Lambent® 703 from Lambent Technologies Inc., and Tegopren® 5840 from Degussa AG.

The use of stabilizers such as ageing inhibitors is known in the plastics industry. For instance, in the production of materials, including materials of construction, comprising PVC or polyolefins, ageing inhibitors are needed in order to protect these plastics from thermal damage during the production operation. In this case use is made sometimes of ageing inhibitor packages consisting of primary and secondary ageing inhibitors (“Plastics Additives Handbook”, “Antioxidants” section, Carl Hanser Verlag, 5^{th} edition).

Surprisingly and foreseeably for the skilled person it has been found that the use of primary ageing inhibitors (sterically hindered phenols or C-radical scavengers, for example) or a combination of primary and the secondary ageing inhibitors (sulphur compounds, phosphites or sterically hindered amines, for example), it also being possible for the functions of the primary and the secondary ageing inhibitor to be united in one molecule, are likewise suitable for the long-term stabilization of the wetting properties and transport properties for biological fluids. The stabilizers used preferably have a molecular weight of more than 500 g/mol and preferably more than 600 g/mol. The amount of and/or sum total of the stabilizers is not more than 1%, based on the sum total of the surfactants in the coating.

A careful selection of the ageing inhibitors is particularly important, since the web material of the invention comes into direct contact with the biological fluid, the analyte. The analyte, or biological fluid to be analysed, is supplied within the diagnostic strip with a specific enzyme for a subsequent detection reaction. There is a risk of the enzyme reactions possibly being inhibited by certain ageing inhibitors. In order to prevent such inhibition and hence the failure in function of the diagnostic strip, it is preferred to use ageing inhibitors having a low solubility in water or in aqueous biological fluid. The solubility of the stabilizer or stabilizers in water is preferably less than 0.2 g/l and more preferably less than 0.05 g/l.

Particularly suitable stabilizers in the coating of the invention are ageing inhibitors based on a secondary aromatic amine, for example Irganox® 5057, on a sterically hindered amine (HALS), for example Tinuvin® 123, on an organic thioether, for example Irganox® PS800, on an organic phosphate ester, for example Irgafos® 168, or, with particular preference, on a sterically hindered phenol, for example Irgano® 245, Irganox® 1010 or Irganox® 1098, all from Ciba Specialty Chemicals Inc. Preference is additionally given to a combination of a sterically hindered phenol with a secondary aromatic amine, a sterically hindered amine (HALS), an organic thioether or an organic phosphite ester.

The coating may be applied to one side of both sides of the carrier material. The surfactant or surfactant mixture is coated from an aqueous, alcoholic, preferably ethanolic, solution or, with particular preference, from an ethanol/water mixture in a weight ratio of 1:9 to 9:1. Coating can be carried out using the typical methods. Examples that may be mentioned include spray coating, engraved-roll application, Mayer bar coating, multi-roll coating, printing techniques such as screen printing, and condensation coating (“Modern Coating and Drying Techn.”, Cohne, 1992, Wiley-VCH, ISBN 0-471-18806-9).

As processing aids for the coating operation it is likewise possible, for the material of the invention, to use defoamers. Solutions containing surfactant tend to form foam in operations in which these solutions are agitated, stirred and/or coated. In the course of such foaming, air (gas) becomes dispersed in the liquid. The surfactants stabilize the foam lamellae and prevent the bubbles from bursting. The function of defoamers is to destabilize these foam lamellae and so to bring about a rapid reduction in the foam, and/or to prevent the formation of foam. Defoamers are generally organic oils or silicone oil, which may contain hydrophobic fillers. When using defoamers it is necessary to ensure that the wetting properties and the ageing stability of the coated material of the invention are not adversely affected. Defoamers which have proven to be particularly effective and compatible with the functional capacity are those based on organically modified siloxanes. Mention may be made here, by way of example, of Tego® Surten A 2-89 and A 288 from Degussa AG, and also Silicone 1520 from Dow Corning Inc.

Used as a basis for the carrier material of the invention are the typical carrier materials familiar to the skilled person, such as films of polyethylene, polypropylene, oriented polypropylene, polyvinyl chloride, polyester and, with particular preference, polyethylene terephthalate (PET). These may be monofilms, coextruded films or laminated films. This enumeration is exemplary and not exhaustive. The surface of the films may have been microstructured by means of suitable techniques such as embossing, etching or laser treatment, for example. The use of laminates, nonwovens, woven fabrics or membranes is likewise possible. For the purpose of improved anchorage of the coating, the carrier materials may have been chemically or physically pretreated by the standard methods, examples of which that may be mentioned include corona treatment or flame treatment. To promote adhesion it is likewise possible to prime the carrier material using, for example, PVC, PVDC or thermoplastic polyester copolymers.

The thickness of the film of the invention is 12 to 350 μm and preferably 50 to 150 μm.

In a channel (80 μm×1.5 mm×15 mm), in which the surfactant-containing coating forms one wall of the channel, in one advantageous development of the invention the transport rate of biological fluids such as blood is at least 3 mm/s.

The coating of the invention affords a good strength of anchorage for the pressure-sensitive adhesive tapes used for the construction of the biosensors and diagnostic strips. In some cases the pressure-sensitive adhesive tapes used combine high shear strength with a low adhesive application rate, such tapes being as disclosed in DE 10 2004 013 699 A1, for example. To prevent delamination of the biosensors and diagnostic strips in the production operation and in use, an effective strength of anchorage of these pressure-sensitive adhesive tapes must be ensured. An effective strength of anchorage is manifested in adhesive-bond assembly strengths (bond strength to the coated surface) of at least 3.5 N/cm and preferably of at least 4.0 N/cm. One adhesive tape which meets this requirement is tesa® 4872, a double-sided pressure-sensitive adhesive tape consisting of a 12 μm PET carrier film coated on both sides with an acrylate adhesive, having a product thickness of 48 μm and a bond strength to steel of 4.0 N/25 mm.

On the basis of the properties outlined, the material of the invention finds application for, among other things, the construction of medical diagnostic strips for analysing biological fluids, also referred to as biosensors. Further applications for the material of the invention are what are called point-of-care devices and microfluidic devices, by which are meant medical instruments, test plates or test strips containing microstructures or microchannels. These microfluidic devices can likewise be used to analyse biological fluids. Examples that may be mentioned include test strips for determining the coagulation behaviour of blood.

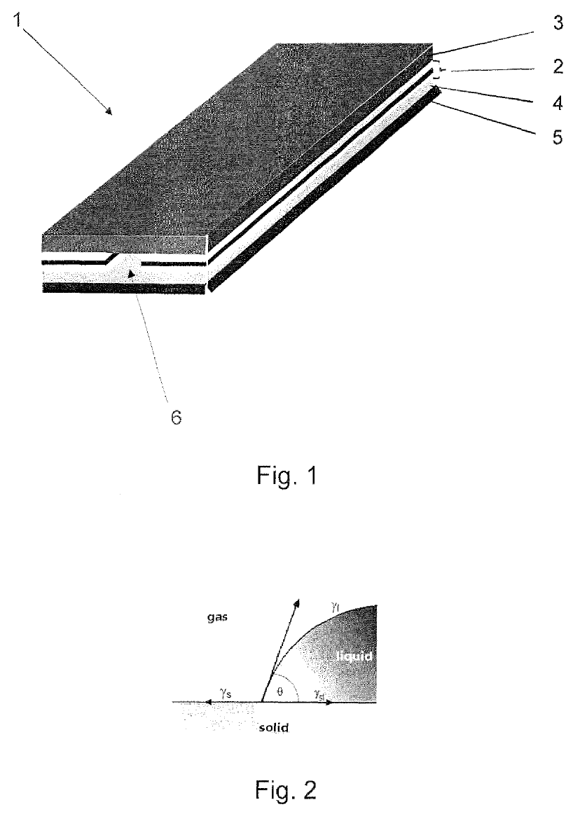

An exemplary construction of a medical diagnostic test strip comprising the material of the invention is depicted diagrammatically in FIG. 1.

Test strip 1 is composed of layers 2, 3, 4 and 5.

Printed onto base material 3 are the electrical conductor tracks and the immobilized enzyme. This printed base layer is for example a diecut of a double-sided pressure-sensitive adhesive tape 2. Tape 2 itself has two layers of, preferably, a pressure-sensitive polyacrylate adhesive between which there is a PET carrier. The diecut of tape 2 forms a measuring channel 6 which is needed to transport the biological fluid to be measured, blood for example, to the measuring cell. Material 5 with hydrophilic coating 4 is laminated onto tape 2 in such a way that this hydrophilic coating faces towards the inside of the measuring channel and thus forms one of its walls.

### Test Methods

### Surface Tension and Contact Angle Measurement

The measurement of the contact angle with water and of the surface tension on solid surfaces takes place in accordance with EN 828:1997 using a G2/G402 instrument from Krüss GmbH. The surface tension is determined by the method according to Owens-Wendt-Rabel & Kaeble, by measuring the contact angle with deionized water and diiodomethane. The values are obtained in each case by averaging four results.

### Static Surface Tension (Liquid) and CMC

The measurement of the surface tension of aqueous fluids takes place in accordance with the ring method (du Nouy) in accordance with DIN EN 14210 using the Tensiometer K100 instrument from Krüss GmbH at 20° C.

The critical micelle concentration (CMC) is determined via the automatic metering of the surfactant by the K100 tensiometer from Krüss GmbH, with subsequent evaluation of the concentration/surface behaviour (DIN 53914).

### Capillary Test

The measurement of the transport rate of biological fluids takes place in a capillary test. For this purpose an uncoated and untreated surface of a PET film 350 μm thick is laminated with two strips of a double-sided adhesive tape having a thickness of 80 μm (tesa® 4980, a double-sided pressure-sensitive tape consisting of a 12 μtm PET carrier film coated on both sides with an acrylate adhesive, product thickness 80 μm, bond strength to steel 19.3 N/25 mm) in parallel. These two adhesive tape strips are laminated in such a way as to form a channel with a width of 1.5 mm exactly. This channel is subsequently covered with the film under test, so that the surface under test forms one wall of the channel. The channel, or capillary, has the following dimensions: height 80 μm, width 1.5 mm and length 5 cm. The capillary is then held upright to a depth of 1 mm in animal blood. Using a stopwatch, a measurement is made of the time needed for the liquid front to cover 15 mm. The result reported for the capillary test is the rate of the blood front in mm/s.

### Areal Spread and Spread Rate

For the measurement of the areal spread, 0.5 ml of deionized water to which 0.1% by weight of the dye methyl blue has been added is applied uniformly to the surface under test, using a calibrated pipette from Eppendorf. By means of millimetre paper located beneath the test specimen, the diameter of the wetted area is determined after approximately 15 seconds, and is used to calculate the wetting area.

In order to determine the spread rate, this measurement is recorded by video camera and the rate is calculated from the wetting area after 5 seconds.

The measurement is made under standardized climatic conditions of 23° C. and 50% r.h.

### Assembly Strength

To determine the strength of the assembly comprising the test film and a standard pressure-sensitive adhesive tape with a very high-shear-strength pressure-sensitive adhesive, the adhesive tape tesa® 4872, a double-sided pressure-sensitive adhesive tape consisting of a 12 μm PET carrier film coated on both sides with an acrylate adhesive, product thickness 48 μm, bond strength to steel 4.0 N/25 mm, is laminated onto the surface under test by being rolled on ten times using a 2 kg roller. Immediately thereafter a measurement is made of the force needed to part the assembly comprising the surface under test and the pressure-sensitive adhesive tape. This is done by pulling the assembly apart at an angle of 180° in a tensile testing machine.

### Ageing Stability

For the assessment of the storage stability, A4 specimens are stored in a drying cabinet at 40° C. for 10 weeks or at 70° C. for 4 weeks. In the course of storage it should be ensured that the coated surface is not covered.

The intention of the text below is to illustrate the invention by means of a number of examples, without wishing thereby to restrict the invention unnecessarily.

### EXAMPLES

### Example 1

The PET film Hostaphan® RN 100 from Mitsubishi Polyesterfilm GmbH, which is 100 μm thick, is corona-pretreated on one side and then coated with a solution consisting of 0.7% by weight of Tego® Surten W111 (alcohol alkoxylates) and 0.1% by weight of Tego® Surten A 2-89 (defoamer) from Degussa AG in water, using an engraved roller, coating taking place in a width of 2000 mm and at a rate of 200 m/min. The coating is dried in a drying tunnel at 120° C.

In the coating operation the surfactant-containing coating solution is distinguished by a very low foam formation propensity, as a result of which very high coating rates can be achieved The wetting properties decrease somewhat over a storage period of 4 weeks at 70° C.

### Example 2

The PET film Hostaphan® RN 100 from Mitsubishi Polyesterfilm GmbH, which is 100 μm thick, is corona-pretreated on one side and then coated with a solution consisting of 0.4% by weight of Licowet® F 40 (a fluorinated surfactant, CAS No. 65545-80-4) from Clariant AG in water, using a 0.1 mm wire doctor blade, coating taking place in a width of 500 mm and at a rate of 20 m/min. The coating is dried in a drying tunnel at 110° C.

The coating exhibits very good wetting behaviour and a very high spread rate for water and aqueous biological fluids such as blood. The coating exhibits good wetting behaviour even over a storage time of 4 weeks at up to 70° C.

### Example 3

In the same way as in Example 2, the PET film is coated with 0.5% by weight of Tegopren® W 5840 from Degussa AG in ethanol/water (25%/75% by volume) and dried. The coating likewise exhibits very good wetting behaviour and a very high spread rate for water and aqueous biological fluids such as blood. These very good wetting properties remain virtually constant over storage periods (4 weeks at 70° C.).

### Example 4

In the same way as in Example 2, the PET film is coated with 0.5% by weight of Tegopren® W 5840 from Degussa AG and 0.0025% by weight of Irganox® 1098 from Ciba Specialty Inc. in ethanol/water (25%/75% by volume) and dried.

The coating again exhibits very good wetting behaviour and a very high spread rate for water and aqueous biological fluids such as blood. These very good wetting properties also do not change even after storage (4 weeks at 70° C.).

Overview of the Results of the Examples:

| | Unit | Example 1 | Example 2 | Example 3 | Example 4 |
|---|---|---|---|---|---|
| Surface coating | | surfactant + defoamer | surfactant | surfactant | surfactant + stabilizer |

Surfactant properties

| | | | | | |
|---|---|---|---|---|---|
| Surfactant | | nonionic | nonionic | nonionic | nonionic |
| Critical micelle concentration | g/l | 0.1 | 0.03 | 0.05 | 0.05 |
| Surface tension of surfactant at | mN/m | 28 | 20 | 23 | 23 |
| 0.1% by weight in aqueous | | | | | |
| solution | | | | | |

Web material properties

| | | | | | |
|---|---|---|---|---|---|
| Anchorage test | N/cm | 4.6 | 4.0 | 5.1 | 5.0 |
| Surface tension | mN/m | 66 | 75 | 73 | 72 |
| Surface tension after 4 weeks | mN/m | 63 | 70 | 71 | 72 |
| at 70° C. | | | | | |
| Contact angle | ° | 19 | 4 | 6 | 6 |
| Contact angle after 4 weeks at | ° | 21 | 6 | 8 | 5 |
| 70° C. | | | | | |
| Areal spread of 0.5 ml of water | cm² | 12 | 20 | 19 | 19 |
| Areal spread of 0.5 ml of water | cm² | 11 | 18 | 18 | 20 |
| after 10 weeks at 40° C. | | | | | |
| Areal spread of 0.5 ml of water | cm² | 10 | 18 | 16 | 19 |
| after 4 weeks at 70° C. | | | | | |
| Spread rate of 0.5 ml of water | mm²/s | 116 | 271 | 257 | 251 |
| Capillary test | mm²/s | 3.2 | 4.5 | 3.7 | 3.6 |

### Counterexamples

### Counterexample 1

As counterexample 1 the commercial PET film Hostaphan RN 100 from Mitsubishi Polyesterfilm GmbH is used, whose surface has not been modified or coated.

With this film a low surface tension is measured. As a result, aqueous fluids do not spread out on this surface. Nor, as a result, are biological fluids transported into the channel in the channel test.

### Counterexample 2

As counterexample 2 the commercial hydrophilic film ARflow 90128 from Adhesives Research is used. This product consists of a PET film coated on one side with a surfactant-containing hot-melt adhesive. This coating is lined with a protective film.

This hydrophilic film exhibits moderate to good properties with respect to the spreading of biological fluids. After storage a reduction in the wettability of the film is observed. Another disadvantage is the waxlike surface, owing to the high surfactant concentration. When the film is employed in multi-layer constructions in which pressure-sensitive adhesive tapes are bonded to this hydrophilic film, this results in a low bond strength and, consequently, in delamination of this adhesive bond. After storage at 70° C. for 4 weeks a reduction in the wetting properties is observed.

### Counterexample 3

As counterexample 3 the commercial product 9971 from 3M Inc. is used. This PET film possesses a surface-active coating on one side. This coating consists of a surfactant-containing PVDC coating.

This hydrophilic film exhibits moderate spreading properties for biological fluids. In this context the hydrophilic surface properties are very inhomogeneous over the area and also from one batch to another.

### Counterexample 4

As counterexample 4 Example 1 from US 2005/0084681 A1 is reproduced. This is done by coating a 100 μm PET film in the laboratory with 0.6% by weight of Dynoll® 604 from Air Products Inc. and 0.2% by weight of Rhodacal® DS10 from Rhodia in an isopropanol/water mixture (70/30) using a wire doctor blade (No. 1), and then drying the coated film at 100° C. for 3 minutes.

This coating exhibits good wetting properties, which are also stable after storage.

Overview of the Results of the Counterexamples:

| | | Counter- | Counter- | Counter- | Counter- |
|---|---|---|---|---|---|
| | Unit | example 1 | example 2 | example 3 | example 4 |
| Surface modification | | — | TPET + surfactant | PVDC + surfactant | surfactant + stabiliser |

Surfactant properties

| | | | | | |
|---|---|---|---|---|---|
| Surfactant | | — | ionic | ionic | nonionic |
| Critical micelle concentration | g/l | — | not known | not known | 0.11 |
| Surface tension of surfactant at | mN/m | — | not known | not known | 27 |
| 0.1% by weight in aqueous | | | | | |
| solution | | | | | |

Web material properties

| | | | | | |
|---|---|---|---|---|---|
| Anchorage test | N/cm | 5.3 | 1.3 | 5.1 | 3.8 |
| Surface tension | mN/m | 46 | 63 | 61–63* | 64 |
| Surface tension after 4 weeks | mN/m | — | 58 | 58–61* | 65 |
| at 70° C. | | | | | |
| Contact angle | ° | 63 | 22 | 23–27* | 21 |
| Contact angle after 4 weeks at | ° | — | 28 | 25–28* | 20 |
| 70° C. | | | | | |
| Areal spread of 0.5 ml of water | cm² | 25 | 9 | ~7* | 10 |
| | | no | | | |
| | | spreading | | | |
| Areal spread of 0.5 ml of water | cm² | — | 7 | ~6* | 11 |
| after 10 weeks at 40° C. | | | | | |
| Areal spread of 0.5 ml of water | cm² | — | 7 | ~6* | 9 |
| after 4 weeks at 70° C. | | | | | |
| Spread rate of 0.5 ml of water | mm²/s | — | 86 | 60–80* | 95 |
| Capillary test | mm²/s | liquid | 2.9 | ~2.5* | 3.0 |
| | | does not | | | |
| | | migrate | | | |
| | | into | | | |
| | | capillary | | | |

*Properties of the coating are not homogeneous over the area.

1. A web form material for a diagnostic strip, composed of a carrier material having on one or both sides a coating comprising at least one surfactant
which is non-ionic,
which possesses a critical micelle concentration (CMC) of 0.1 g/l or less, and
whose 0.1% strength by weight aqueous solution has a surface tension of <30 mN/m.

2. Web material according to claim 1, wherein the coating possesses the following properties:
a surface tension of at least 65 mN/m
a contact angle with water of less than 20°
an areal spread with 0.5 ml of water of at least 12 cm² and/or
a spread rate of at least 100 mm²/s.

3. Web material according to claim 1 wherein the coating comprises a surfactant having a critical micelle concentration (CMC) of less than 0.08 g/l.

4. Web material according to claim 1, wherein the coating comprises a surfactant whose 0.1% strength by weight aqueous solution has a surface tension of <25 mN/m.

5. Web material according to claim 1, wherein 0.5 ml of water on the coating spreads at least 15 cm² spreads at a rate of at least 150 mm²/s, and/or the value for the contact angle with water on the coating is less than 10°.

6. Web material according to claim 2, wherein the areal spread of 0.5 ml of water on the coating after storage at 40° C. for 10 weeks or at 70° C. for 4 weeks is at least 85% of the original value prior to storage.

7. Web material according to claim 2, wherein the coating comprises a surfactant based on an organically modified siloxane.

8. Web material according to claim 1, wherein the coating is composed of at least one surfactant and at least one ageing inhibitor as stabilizer, the percentage fraction of the stabilizer or stabilizers not exceeding 1% by weight, based on the sum total of the surfactants.

9. Web material according to claim 8, wherein the coating comprises at least one primary ageing inhibitor or a combination of one primary and one secondary ageing inhibitor, the functions of the primary and the secondary ageing inhibitor optionally being united in one molecule.

10. Web material according to claim 8, wherein said at least one ageing inhibitor is based on a secondary aromatic amine, a sterically hindered amine (HALS), an organic thioether, an organic phosphite ester, on a sterically hindered phenol or on a sterically hindered phenol in combination with one or more of said secondary aromatic amine, sterically hindered amine (HALS), organic thioether or organic phosphite ester.

11. Web material according to claim 8, wherein the molecular weight of said at least one ageing inhibitor is at least 500 g/mol or the solubility in water of the stabilizer or stabilizers used in the coating is less than 0.2 g/l or wherein said at least one stabilizer has both said molecular weight and said solubility.

12. Web material according to claim 1, wherein said coating comprises at least one defoamer.

13. Web material according to claim 12, wherein the coating is composed exclusively of a surfactant based on an organically modified siloxane and of a defoamer.

14. Web material according to claim 1, wherein the carrier material is composed of a polyester film having a thickness of 12 to 350 μm.

15. Web material according to claim 1 wherein, in a channel having the dimensions 80 μm×1.5 mm×15 mm and in which the surfactant-containing coating forms one wall of the channel, the transport rate of blood is at least 3 mm/s.

16. Diagnostic strips, biosensors, point-of-care devices or microfluidic devices used to analyse biological fluids, comprising the web material of claim 1.

17. The web material of claim 3, wherein said critical micelle concentration is less than 0.6 g/l.

18. The web material of claim 5, wherein 0.5 ml of water on the coating spreads at least 17 cm² and water on the coating spreads at a rate of at least 200 mm²/s.

19. The web material of claim 7, wherein said coating is composed exclusively of a surfactant based on an organically modified siloxane.

20. The web material of claim 14, wherein said thickness is 50 to 150 μm.
